(19) United States
(12) Patent Application Publication
(10) Pub. No.: US 2005/0037432 A1
(43) Pub. Date: Feb. 17, 2005

(54) Novel biomarkers of aggrecanase activity

(75) Inventors: Mickey Tortorella, O'Fallon, MO (US); Elizabeth Arner, Wildwood, MO (US); Anne-Marie Malfait, St. Louis, MO (US)

Correspondence Address: PHARMACIA CORPORATION;GLOBAL PATENT DEPARTMENT, POST OFFICE BOX 1027, ST. LOUIS, MO, 63006, US

(73) Assignee: Pharmacia Corporation

(21) Appl. No.: 10/900,680

(22) Filed: Jul. 28, 2004

Related U.S. Application Data
(60) Provisional application No. 60/490,564, filed on Jul. 28, 2003.

(51) Int. Cl. G01N 33/53; C07H 21/04; C12N 9/64; C07K 16/40

(52) U.S. Cl. 435/7.1; 435/69.1; 435/226; 435/320.1; 435/325; 530/388.26; 536/23.2

(57) ABSTRACT

Biomarkers for detecting aggrecanas-1 and/or aggrecanase-2 activity are disclosed. The biomarkers are specific peptide fragments of α2 macroglobulin.

This application claims priority to U.S. Provisional application No. 60/490,564, filed Jul. 28, 2003.

### BACKGROUND OF THE INVENTION

The ADAMs (α disintegrin and metalloprotease) are a family of multidomain proteins with structural homology to snake venom metalloproteases. The archetypical ADAM protein has a prodomain, metalloprotease domain, disintegrin domain, and a cysteine-rich region. At least 18 of the ADAMs have now been identified and sequenced, and together with snake venom metalloproteases, make up the reprolysin family of zinc metalloproteases.

Some members of the ADAM family contain thrombospondin motifs, and are referred to as ADAMTS (α disintegrin and metalloprotease with thrombospondin motifs). In particular, ADAMTS-4 and ADAMTS-5 have been shown to cleave aggrecan, and are thus believed to be responsible for the onset or progression or degenerative conditions characterized by degradation of aggrecan, especially aggrecan found in cartilage. ADAMTS-4 is also known as aggrecanase-1, and ADAMTS-5 is also called aggrecanase-2.

Because ADAMTS-4 and ADAMTS-5 are implicated in aggrecan degradation, these proteinases are reasonable targets for therapeutic drugs to reduce or abolish the deleterious effects of these aggrecanases. In this regard, it would be useful to find biomarkers indicating aggrecanase activity. Since aggrecan is a substrate for ADAMTS-4 and ADAMTS-5, it would seem reasonable to use cleaved fragments of aggrecan as biomarkers. Unfortunately, aggrecan and its cleaved fragments are highly glycosylated, making it unsuitable for traditional detection methods such as enzyme linked immunosorbent assay (ELISA) without first removing the sugar residues. This subsequent deglycosylation step is both difficult and time consuming, limiting the value of using aggrecan fragments as biomarkers.

Alpha-2-Macroglobulin (α2M) is a homotetramer of about 720 kDa, and is a general endoproteinase inhibitor circulating in blood at concentrations of 0.6-2 mg/ml, constituting about 2-4% of total plasma protein in humans. α2M is also present in synovial fluid. In its nascent form (SEQ ID NO. 1), α2M is about 1474 residues long. In the mature form (SEQ ID NO. 2) of α2M, the initial signal sequence of about 23 residues (SEQ ID NO. 3) is absent.

α2M is able to inhibit all four classes of proteinases by a unique trapping mechanism. Each subunit of the α2M molecule contains a region referred to as the “bait region,” a short peptide stretch (about 39 amino acid residues in human α2M, SEQ ID NO. 4, from about residue 667 to about residue 706 of the mature protein) that is very susceptible to proteolytic cleavage by specific cleavage sites for different proteinases. When a proteinase cleaves the bait region, a conformational change is induced in α2M, which traps the proteinase. Following cleavage in the bait region a thiolester bond is hydrolyzed and mediates the covalent binding of α2M to the proteinase. The entrapped enzyme remains active against low molecular weight substrates, but activity against high molecular weight substrates is greatly reduced due to steric interference. Because the complex formation of proteinases with α2M is dependent on their proteolytic activities against the bait region, α2M is a useful tool for identifying unknown proteinases. (Nagase, H., Itoh, Y., and Binner, S. (1994) Ann. N.Y. Acad. Sci. 732, 294-302). Indeed, some members of the ADAM and ADAMTS family of enzymes have been characterized by their inhibition by α2M.

### SUMMARY OF THE INVENTION

It has been found that α2M is an endogenous inhibitor of the cartilage aggrecanases, ADAMTS-4 and ADAMTS-5. Both ADAMTS-4 and ADAMTS-5 cleave α2M in the bait region between amino acids Met⁶⁹⁰ and Gly⁶⁹¹, generating the C-terminal neoepitope YESDVM⁶⁹⁰ (Tyr-Glu-Ser-Asp-Val-Met⁶⁹⁰) (SEQ ID NO. 6) and the N-terminal neoepitope ⁶⁹¹GRGHAR (⁶⁹¹Gly-Arg-Gly-His-Ala-Arg) (SEQ ID NO. 7). These cleavage sites in α2M are believed to be unique to aggrecanases. The present invention relates to these novel neoepitopes, and methods of detecting them. The present invention also provides for a method of determining the efficacy of aggrecanase inhibiting compounds, preferably aggrecanase inhibiting pharmaceutical compositions.

### DETAILED DESCRIPTION OF THE INVENTION

Antibodies were designed, using techniques well known in the art, to the new C-terminus SDVM⁶⁹⁰ (SEQ ID NO. 8) and the N-terminus ⁶⁹¹GRGH (SEQ ID NO. 9) following specific cleavage of α2M at the Met⁶⁹⁰/Gly⁶⁹¹ bond by ADAMTS-4 and ADAMTS-5. Techniques to raise antibodies to neoepitopes formed by cleavage of collagen are described, for example, in U.S. Pat. No. 6,030,792, issued Feb. 29, 2000 to Otterness et al., the disclosure of which is incorporated herein by reference. The techniques of the present invention may be conducted in a similar fashion. In order to test the ability of these antibodies to detect ADAMTS-4 and ADAMTS-5 cleavage products, α2M (50 ng) was digested with 100 ng of ADAMTS-4 and ADAMTS-5 for varying periods of time (1 min, 2 min, 3 min, 5 min, 10 min, 15 min, and 30 min) at 37° C. Following the incubations the products were analyzed for anti-SDVM⁶⁹⁰ and anti-⁶⁹¹GRGH. Both neoepitopes were generated as early as after 2 minutes of incubation, and increased over time, up to after 30 minutes of incubation. Generation of the neoepitopes, as detected with these neoepitope antibodies, was inhibited in the presence of EDTA or in the presence of an aggrecanase-inhibitor: 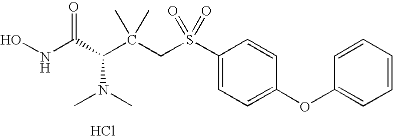
2-dimethylamino-N-[1-(hydroxyamino)-carbonyl]-3,3-dimethyl-4-[(4-phenoxyphenyl)-sulphonyl]-butane.

The neoepitope antibodies anti-SDVM⁶⁹⁰ and anti-⁶⁹¹GRGH were used to detect fragments of α2M in synovial fluids of patients with osteoarthritis. Preliminary experiments suggest the presence of the neoepitope SDVM⁶⁹⁰ (SEQ ID NO. 8) in the synovial fluid of patients with osteoarthritis.

These findings suggest that the neoepitopes SDVM⁶⁹⁰ (SEQ ID NO. 8) and ⁶⁹¹GRGH (SEQ ID NO. 9), generated when α2M is cleaved by aggrecanases, are biomarkers for aggrecanase activity in arthritis or other conditions where pathological expression of aggrecanases are implicated. These neoepitopes are generated during the osteoarthritic process as illustrated by their detection in synovial fluid. Therefore, they are expected to be detectable in blood or urine as convenient markers of aggrecanase activity and of the ability of aggrecanase inhibitors to block this increased activity in disease. By detecting aggrecanase activity prior to gross manifestation of disease, such as overt tissue damage, it may be possible to arrest or slow the onset of aggrecanase mediated diseases at an early stage of such diseases.

While the detection method employed in the foregoing examples was an ELISA assay, it will be appreciated that any appropriate assay could be employed for detection of the neoepitopes described. For example, mass spectroscopy could be used to detect the various neoepitopes.

Other variations will occur to those skilled in the art in light of the foregoing disclosure. For example, the detection and monitoring of disease conditions other than osteoarthritis may find use in the methods of the present invention. Peptides that contain the sequences described may be prepared synthetically. Nucleotide sequences coding for the sequences described may be prepared for expression of such peptides. mRNA sequences may be prepared for detection of or translation of such peptides as well. All embodiments herein are merely exemplary, not limitative, and thus variations are within the intended scope of the appended claims.

1. A peptide comprising an N terminal end consisting of the sequence: Gly Arg Gly His (SEQ ID NO. 9).

2. A peptide comprising a C terminal end consisting of the sequence: Ser Asp Val Met (SEQ ID NO. 8).

3. A peptide comprising an N terminal end consisting of the sequence: Gly Arg Gly His Ala Arg (SEQ ID NO. 7).

4. A peptide comprising a C terminal end consisting of the sequence: Tyr Glu Ser Asp Val Met (SEQ ID NO. 6).

5. A sequence selected from the group consisting of: Ser Asp Val Met Gly Arg Gly His (SEQ ID NO. 5);
Tyr Glu Ser Asp Val Met (SEQ ID NO. 6);
Gly Arg Gly His Ala Arg (SEQ ID NO. 7);
Ser Asp Val Met (SEQ ID NO. 8); and
Gly Arg Gly His (SEQ ID NO. 9).

6. An antibody whose epitope is selected from the group consisting of:
Ser Asp Val Met Gly Arg Gly His (SEQ ID NO. 5);
Tyr Glu Ser Asp Val Met (SEQ ID NO. 6);
Gly Arg Gly His Ala Arg (SEQ ID NO. 7);
Ser Asp Val Met (SEQ ID NO. 8); and
Gly Arg Gly His (SEQ ID NO. 9).

7. A method of detecting aggrecanase activity in a mammal comprising:
detecting in a fluid or tissue from said mammal a sequence selected from the group consisting of:
Ser Asp Val Met Gly Arg Gly His (SEQ ID NO. 5);
Tyr Glu Ser Asp Val Met (SEQ ID NO. 6);
Gly Arg Gly His Ala Arg (SEQ ID NO. 7);
Ser Asp Val Met (SEQ ID NO. 8); and
Gly Arg Gly His (SEQ ID NO. 9).

8. A method of determining the efficacy of a compound in inhibiting aggrecanase comprising:
detecting in a fluid or tissue a peptide comprising a sequence selected from the group consisting of:
Ser Asp Val Met Gly Arg Gly His (SEQ ID NO. 5);
Tyr Glu Ser Asp Val Met (SEQ ID NO. 6);
Gly Arg Gly His Ala Arg (SEQ ID NO. 7);
Ser Asp Val Met (SEQ ID NO. 8); and
Gly Arg Gly His (SEQ ID NO. 9);
treating said fluid or tissue with a compound;
detecting in said fluid or tissue a sequence selected from the group consisting of:
Ser Asp Val Met Gly Arg Gly His (SEQ ID NO. 5);
Tyr Glu Ser Asp Val Met (SEQ ID NO. 6);
Gly Arg Gly His Ala Arg (SEQ ID NO. 7);
Ser Asp Val Met (SEQ ID NO. 8); and
Gly Arg Gly His (SEQ ID NO. 9), after administration of said compound; and
comparing the relative amount of said sequences prior to said treatment with said compound, and subsequent to said treatment with said compound.


SEQUENCE LISTING (9 sequences)

SEQ ID NO: 1 - protein, 1474 aa, Homo sapiens
  ref <308>: DATABASE ACCESSION NUMBER: SWISSPROT/P01023
  ref <309>: DATABASE ENTRY DATE: 1996-06-01
  ref <313>: RELEVANT RESIDUES: (1)..(1474)
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 2 - protein, 1450 aa, Homo sapiens
SVSGKPQYMVLVPSLLHTETTEKGCVLLSYLNETVTVSALESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENELANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEZNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYIKIALLEIPLTVTHPVVRNALFCLESAWKTAEEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDKVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 3 - protein, 23 aa, Homo sapiens
MGKNKLLHPSLVLLLLVLLPTDA

SEQ ID NO: 4 - protein, 39 aa, Homo sapiens
PQLQQYEMHGPEGLRVGFYESDVMGRGHARLVHVEEPHT

SEQ ID NO: 5 - protein, 8 aa, Homo sapiens
SDVMGRGH

SEQ ID NO: 6 - protein, 6 aa, Homo sapiens
YESDVM

SEQ ID NO: 7 - protein, 6 aa, Homo sapiens
GRGHAR

SEQ ID NO: 8 - protein, 4 aa, Homo sapiens
SDVM

SEQ ID NO: 9 - protein, 4 aa, Homo sapiens
GRGH